(19) United States
(12) Patent Application Publication
(10) Pub. No.: US 2026/0008752 A1
(43) Pub. Date: Jan. 8, 2026

(54) POLYMORPHIC FORMS OF FLORBETAPIR PRECURSOR AV-105

(71) Applicant: ELI LILLY AND COMPANY, Indianapolis, IN (US)

(72) Inventor: Adrian Samuel VELLEKOOP, Altamont, NY (US)

(21) Appl. No.: 19/129,405

(22) PCT Filed: Nov. 13, 2023
(86) PCT No.: PCT/US2023/079465

§ 371 Date: May 13, 2025

Related U.S. Application Data
(60) Provisional application No. 63/425,091, filed on Nov. 14, 2022.

(51) Int. Cl. C07D 213/64; A61K 31/44; C07B 59/00

(52) U.S. Cl. CPC C07D 213/64; A61K 31/44; C07B 59/002

(57) ABSTRACT

New polymorphic crystal forms of the following compound are disclosed:

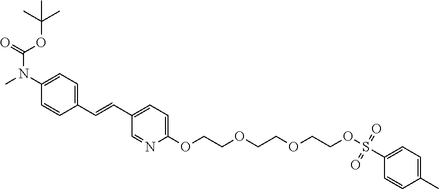 [SMILES: Cc1ccc(S(=O)(=O)OCCOCCOCCOc2ccc(/C=C/c3ccc(N(C)C(=O)OC(C)(C)C)cc3)cn2)cc1]

This compound is used to make florbetapir (¹⁸F).

The present invention relates to a novel polymorph of a precursor used to make florbetapir, to pharmaceutical compositions comprising the precursor, to methods of using the precursor to make florbetapir.

Alzheimer's Disease (AD) is a neurodegenerative disorder characterized by the formation of deposits in the brain containing amyloid-β (“Aβ”). Detection of these deposits by histological examination post mortem has been used to confirm a diagnosis of AD. See U.S. Pat. No. 9,592,308 and WO 2009/059977.

PET imaging has significantly advanced the diagnosis of AD in living patients. In PET imaging, a positron-emitting radioisotope is introduced into a compound that specifically binds a target molecule. Due to its half-life of about 110 minutes, ¹⁸F is a commonly used radioisotope in PET. For AD, one of the targets of most interest for PET is Aβ. Specifically, using PET imaging to examine amyloid load in the brain is an important tool for patient stratification and treatment monitoring.

One commercially available PET radiopharmaceutical for amyloid imaging is florbetapir. Florbetapir is available commercially under the tradename Amyvid®. This agent was approved by the FDA in 2012 to estimate Aβ plaque density in adult patients. Florbetapir is administered to the patient, and then a PET scan is taken as a means of showing the doctor the amyloid burden in the patient's brain.

¹⁸F-Florbetapir is (E)-4-(2-(6-(2-(2-(2[¹⁸F]fluoroethoxy)ethoxy)ethoxy)pyridine-3-yl)vinyl-N-methylbenzamine and has the following structure:

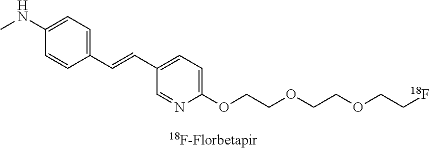 [SMILES: CNc1ccc(/C=C/c2ccc(OCCOCCOCC[18F])nc2)cc1]

Florbetapir is described in U.S. Pat. Nos. 7,687,052 and 8,506,929:

The commercial distribution of ¹⁸F-labeled radiopharmaceuticals (including florbetapir) is complicated by the short half-life of the radioisotope. Specifically, once the supply is made, it must be administered to the patient within about 10 hours. Thus, in some instances, the radiopharmaceutical supplier will actually provide a precursor molecule to the PET imaging center, which the PET imaging center will convert into florbetapir, which can then be quickly administered to the patient so that the PET scan may be taken.

One precursor molecule for florbetapir has the following chemical structure and is referred to herein as the compound of Formula I:

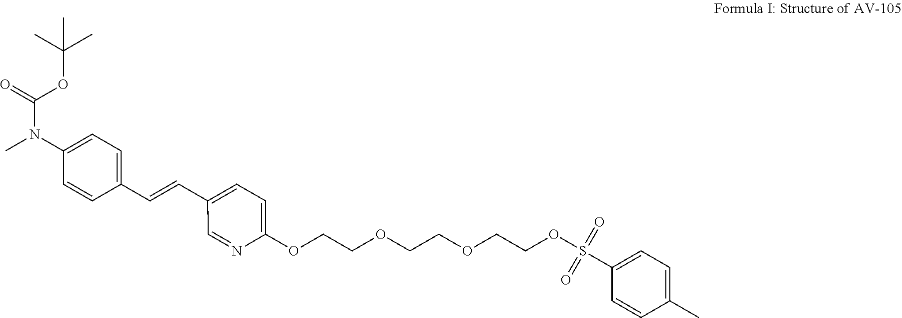 [SMILES: Cc1ccc(S(=O)(=O)OCCOCCOCCOc2ccc(/C=C/c3ccc(N(C)C(=O)OC(C)(C)C)cc3)cn2)cc1]

This molecule is also know as “AV-105”. AV-105, as well as the method of using AV-105 to make ¹⁸F-florbetapir is known in the literature. See John Lister-James, Michael J Pontecorvo, Chris Clark, Abhinay D Joshi, Mark A Mintun, Wei Zhang, Nathaniel Lim, Zhiping Zhuang, Geoff Golding, Seok Rye Choi, Tyler E Benedum, Paul Kennedy, Franz Hefti, Alan P Carpenter, Hank F Kung, Daniel M Skovronsky, “Florbetapir f-18: a histopathologically validated Beta-amyloid positron emission tomography imaging agent,” Semin Nucl Med. 2011 July;41(4):300-4.

AV-105 is commercially available and can also be synthesized by those skilled in the art, for example, by using the techniques (and/or similar techniques) to what is found in U.S. Pat. Nos. 7,687,052 and 8,506,929.

There is a need for alternative solid-state forms of florbetapir precursors with improved thermodynamic stability for the manufacturing of the active pharmaceutical product and drug products.

### BRIEF DESCRIPTION OF THE DRAWINGS

FIG. 1 depicts the XRPD pattern of AV-105 Form A (collected with Cu-Kα radiation).

FIG. 2 depicts the XRPD pattern of AV-105 Form B (collected with Cu-Kα radiation).

FIG. 3 depicts the XRPD patterns of AV-105 Form B+small peaks as described in Example 3. The top pattern is the pattern corresponding to the Form B+small peaks, wherein the arrows indicate the extra peaks that are not present in pure Form B (bottom pattern).

FIG. 4 depicts a comparison of DSC thermograms for AV-105 Form A (top line) and Form B (bottom line).

### DETAILED DESCRIPTION

### Form A

The present embodiments provides a crystalline form of a compound of Formula I, and is characterized by an X-ray powder diffraction pattern using CuKα radiation comprising a peak at diffraction angle 2-theta of 20.9° and one or more peaks selected from the group consisting of 3.8°, 15.1° and 21.2°; with a tolerance for the diffraction angles of ±0.2 degrees.

In some embodiments, the crystalline form of the compound of Formula I is characterized by the X-ray powder diffraction pattern using CuKα radiation comprising a peak at diffraction angle 2-theta of 20.9° and a peak at 3.8°.

In other embodiments, the crystalline form of a compound of Formula I is characterized by an X-ray powder diffraction pattern using CuKα radiation comprising a peak at diffraction angle 2-theta of 20.9° and a peak at 15.1°.

In further embodiments, the crystalline form of the compound of Formula I is characterized by an X-ray powder diffraction pattern using CuKα radiation comprising a peak at diffraction angle 2-theta of 20.9° and a peak at 21.2°.

In further embodiments, the crystalline form of the compound of Formula I is characterized by an X-ray powder diffraction pattern using CuKα radiation comprising a peak at diffraction angle 2-theta of 20.9° and one or more peaks selected from the group consisting of 3.8°, 15.1° and 21.2°, wherein the X-ray powder diffraction pattern using CuKα radiation further comprises a peak at diffraction angle 2-theta of 11.3°; with a tolerance for the diffraction angles of ±0.2 degrees.

The present embodiments also provide the compound of Formula I which is crystalline and is characterized by an X-ray powder diffraction pattern using CuKa radiation comprising two peak at diffraction angle 2-theta, wherein the two peaks are selected from the group consisting of 3.8, 7.5, 11.3, 15.1, 15.7, 16.8, 18.7, 19.1, 20.9, and 21.2; with a tolerance for the diffraction angles of 0.2 degrees.

The present embodiments also provide the compound of Formula I which is a crystalline form of AV-105 and is characterized by an X-ray powder diffraction pattern using CuKα radiation comprising a peak at a diffraction angle 2-theta of 11.3° and one more peaks selected from the group consisting of 3.8° and 7.5°; with a tolerance for the diffraction angles of ±0.2 degrees.

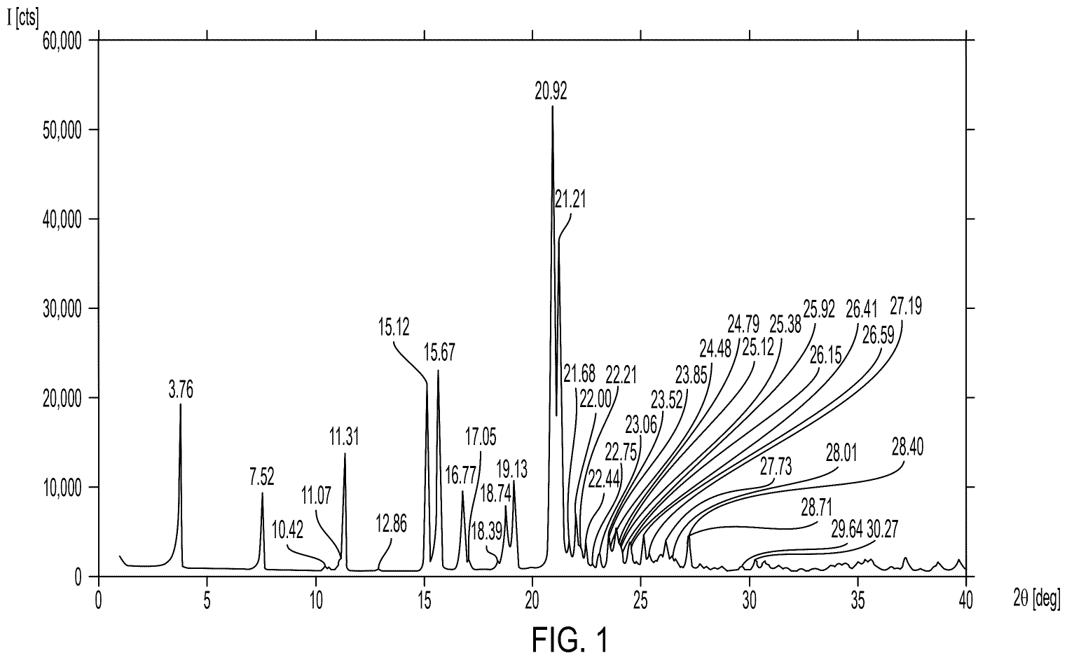

In further embodiments, the crystalline form of the compound of Formula I is characterized by an X-ray powder diffraction pattern using CuKα radiation comprising peaks depicted in FIG. 1.

### Form B

The present embodiments provide a crystalline form of a compound of Formula I, and is characterized by an X-ray powder diffraction pattern using CuKα radiation comprising a peak at diffraction angle 2-theta of 20.7° and one or more peaks selected from the group consisting of 13.3° and 19.4°; with a tolerance for the diffraction angles of ±0.2 degrees.

In other embodiments, the crystalline form of a compound of Formula I is characterized by an X-ray powder diffraction pattern using CuKα radiation comprising a peak at diffraction angle 2-theta of 20.7° and a peak at 13.3°.

In other embodiments, the crystalline form of a compound of Formula I is characterized by an X-ray powder diffraction pattern using CuKα radiation comprising a peak at diffraction angle 2-theta of 20.7° and a peak at 19.4°.

The present embodiments also provide the compound of Formula I which is crystalline and is characterized by an X-ray powder diffraction pattern using CuKα radiation comprising a peak at diffraction angle 2-theta of 20.7° and one or more peaks selected from the group consisting of 12.7°, 13.3°, 17.8°, 19.4° and 23.7°; with a tolerance for the diffraction angles of ±0.2 degrees.

The present embodiments also provide the compound of Formula I which is crystalline and is characterized by an X-ray powder diffraction pattern using CuKα radiation comprising two peak at diffraction angle 2-theta, wherein the two peaks are selected from the group consisting of 9.0, 9.2, 10.3, 12.7, 13.3, 13.5, 17.8, 18.9, 19.4, 20.7, 22.7, 23.7, and 27.6; with a tolerance for the diffraction angles of ±0.2 degrees.

The present embodiments provides a crystalline form of a compound of Formula I, and can be characterized by an X-ray powder diffraction pattern using CuKα radiation comprising a peak at a diffraction angle 2-theta of 13.3° and at least one additional peak selected from the group consisting of 13.5°, 9.2° and 19.4°; with a tolerance for the diffraction angles of ±0.2 degrees.

In further embodiments, the crystalline form of the compound of Formula I is characterized by an X-ray powder diffraction pattern using CuKα radiation comprising a peak at diffraction angle 2-theta of 13.3° and a peak at 13.5°.

In further embodiments, the crystalline form of the compound of Formula I is characterized by an X-ray powder diffraction pattern using CuKα radiation comprising a peak at diffraction angle 2-theta of 13.3° and a peak at 19.4°.

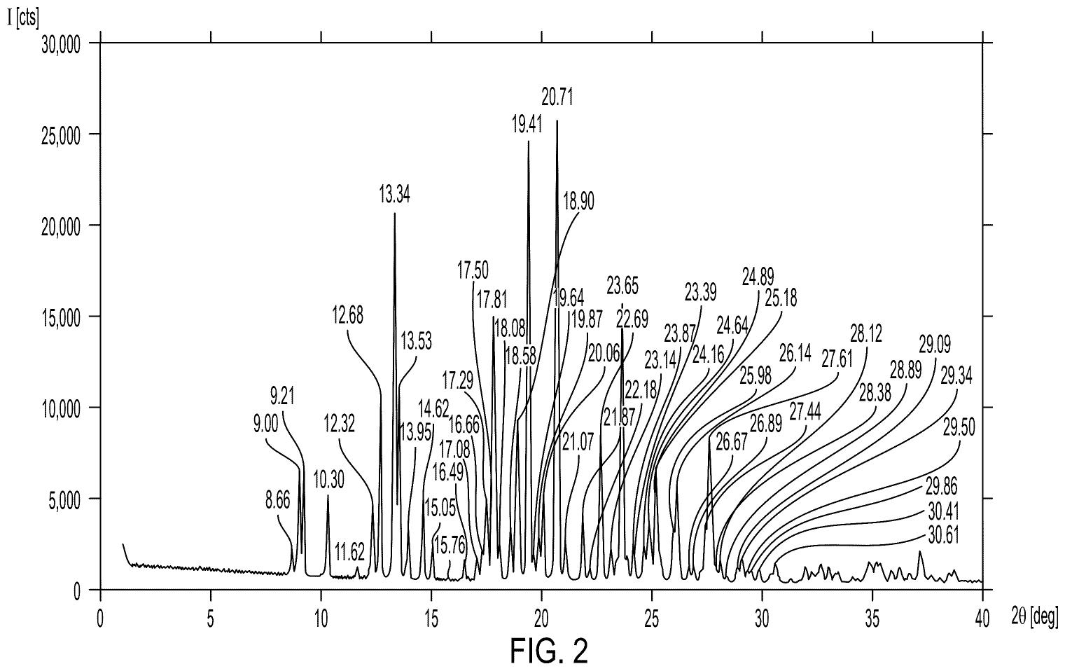

In further embodiments, the crystalline form of the compound of Formula I is characterized by an X-ray powder diffraction pattern using CuKα radiation comprising peaks depicted in FIG. 2.

The present invention further provides a florbetapir precursor to pharmaceutical composition comprising a compound of Formula I. In a particular embodiment, the composition further comprises a recrystallization for AV-105 Form B.

The present invention provides a pharmaceutical composition comprising any compound of the present disclosure and one or more pharmaceutically acceptable excipients. In some embodiments, the pharmaceutical composition comprises a crystalline form of AV-105 and one or more pharmaceutically acceptable excipients. In some embodiments, the pharmaceutical composition comprises a Form A of AV-105 and one or more pharmaceutically acceptable excipients. In some embodiments, the pharmaceutical composition comprises a Form B of AV-105 and one or more pharmaceutically acceptable excipients.

Furthermore, the present invention provides a compound of Formula I for use as a precursor for florbetapir therapy. Any of the recited compounds may be used. In some embodiments, the present invention provides a compound of Formula I for use as a precursor for use in PET diagnostic imaging. Any of the recited compounds may be used.

In addition, the present invention provides the use of a compound of Formula I for the manufacture of a precursor to a medicament for the treatment or diagnosis of a disease or condition selected from AD or other disease associated with build-up of amyloid-beta. Any of the recited compounds may be used.

The present embodiments also include any of the compounds (and polymorphs) listed above for use as a precursor for ¹⁸F-florbetapir.

The present embodiments also include any of the compounds (and polymorphs) listed above for use as a precursor for use as a precursor for ¹⁸F-florbetapir therapy.

The present embodiments also include any of the compounds (and polymorphs) listed above for use as a precursor for use in PET diagnostic imaging.

The present embodiments include a method of making ¹⁸F-florbetapir comprising reacting any of the compounds (and polymorphs) listed above with an ¹⁸F source.

The present embodiments include a method of preparing any of the compounds (and polymorphs) as described herein.

The present embodiments also include the use of any of the compounds (and polymorphs) listed above for the manufacture of a precursor to a medicament for the treatment or diagnosis of a disease or condition selected from AD or other disease associated with build-up of amyloid-beta.

This invention also encompasses polymorphs of the compound of Formula I that are more thermodynamically stable and provide better and/or more reliable properties than prior crystalline forms.

### ABBREVIATIONS AND DEFINITIONS

| | |
|---|---|
| Term | Definition |

| aq | aqueous | |
|---|---|---|
| Å | angstrom | |
| BOC/Boc | tert-butyloxycarbonyl | |
| CPME | cyclopropyl methyl ether | |
| DCM | dichloromethane | |
| DMF | dimethylformamide | |
| EtOAc | ethyl acetate | |
| EtOH | Ethanol | |
| equiv | equivalent | |
| HEP | heptane | |
| IPA | isopropanol | |
| IPC | In-process control | |
| IPrOAc | Isopropyl acetate | |
| kg | kilogram | |
| KOtBu | potassium tert-butoxide | |
| MeOH | methanol | |
| MIBK | methyl isobutyl ketone | |
| min | minute | |
| mL | milliliter | |
| mm | millimeter | |
| mol | mole | |
| MTBE | methyl tert-butyl ether | |
| MW | molecular weight | |
| μL | microliter | |
| μm | micrometer | |
| NMR | nuclear magnetic resonance | |
| OTs | O-para-toluenesulfonate | |
| Pd(OAc)₂ | palladium acetate | |
| PET | positron emission tomography | |
| ppm | parts per million | |
| TEA | triethylamine | |
| vol | volume | |
| wt | weight | |
| XRPD | X-ray powder diffraction | |
| ‘—’ | In tables: | No entry/information |
| | Impurity reporting: | Peak not observed |

### Example 1. Method of Manufacture for AV-105 Form B

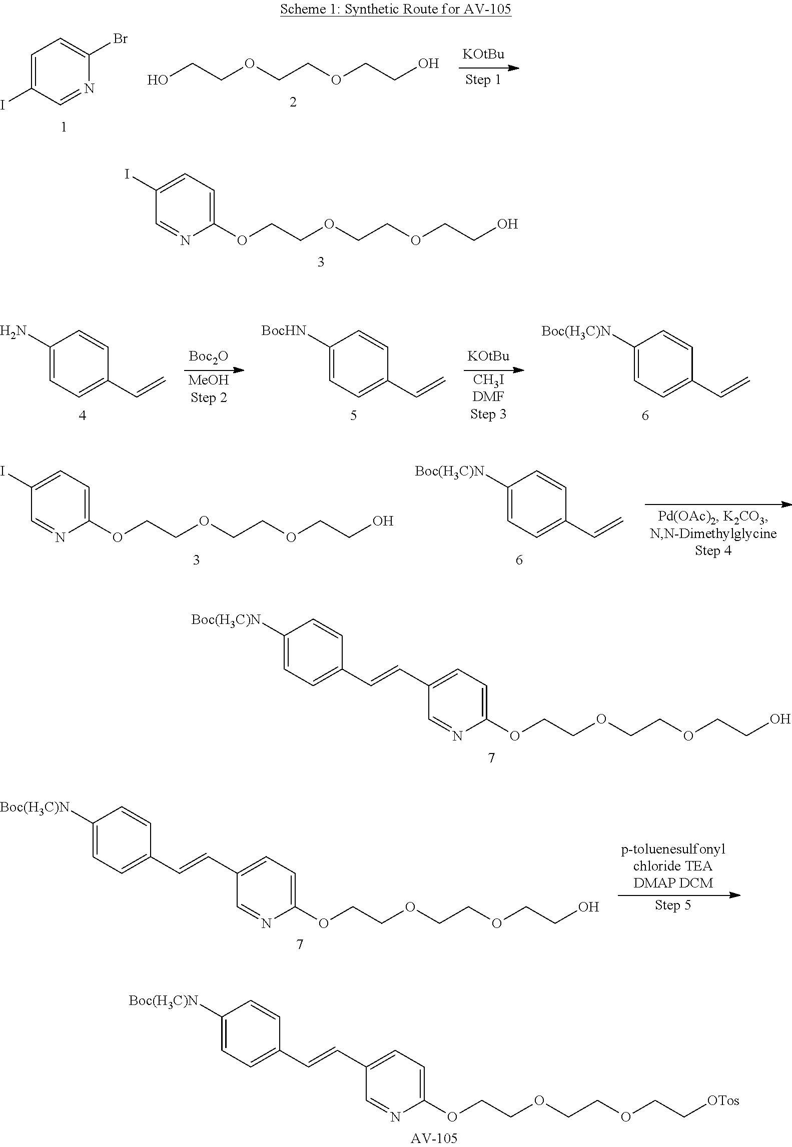

The synthesis of AV-105 is a five-step process. Following the prophetic example, this is the synthetic method for Step 5:

Compound 7 (basis material, 1.00 equiv.) is reacted with para-toluenesulfonyl chloride (pTsC1, 1.20 mol equiv.), triethylamine (TEA, 1.25 mol equiv.), and catalytic N,N-dimethylaminopyridine (DMAP, 0.0500 mol equiv.) in dichloromethane (DCM, 5.0 vol.) at 25° C.; the reaction is quenched with water (2.0 vol.) and crude AV-105 isolated by extractive work-up from DCM-water. Crude AV-105 is purified by column chromatography on silica gel using ethyl acetate-heptane gradient. AV-105 column fractions meeting purity criteria are combined and concentrated. AV-105 Precursor is re-crystallized from methanol with seeding employing AV-105 Form A, is filtered, is washed with methanol, and is dried.

A process flowchart for Step 5 of the current process to AV-105 Form A is provided below in Scheme 2 and Scheme 3:

Following prophetic example, this is the Recrystallization Protocol for Form A

AV-105 (basis material, 1.00 equiv.) dissolved in MeOH (3.6 vol.) at 40±3° C. and passed through a 0.45-μm in-line filter. The system is rinsed with MeOH (3.6 vol.) and the temperature adjusted to 15-20° C. The mixture is seeded with AV-105 Form A (1.0 wt % slurry in 0.025 vol. MeOH) with agitaion at 15-20° C. for 30-45 min. The temperature is adjusted to −20±3° C. (target 5° C./10 min.) and held for 1-20 hours before filtration.

### Example 1A. XRPD Data of AV-105 Form A

XRPD patterns were collected with a PANalytical X'Pert PRO MPD or Empyrean diffractometers using an incident beam of Cu radiation produced using an Optix long, fine-focus source. An elliptically graded multilayer mirror was used to focus CuKα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5. The data acquisition parameters for each pattern are displayed above the image in the Data section of this report including the divergence slit (DS) before the mirror.

TABLE 1
Observed peaks for AV-105 Form
A for XRPD with CuKα radiation.

| °2θ | Intensity (%) |
|---|---|
| 3.8 | 37 |
| 7.5 | 17 |
| 10.4 | 2 |
| 11.1 | 4 |
| 11.3 | 26 |
| 12.9 | 2 |
| 15.1 | 40 |
| 15.7 | 45 |
| 16.8 | 18 |
| 17.1 | 3 |
| 18.4 | 3 |
| 18.7 | 15 |
| 19.1 | 20 |
| 20.9 | 100 |
| 21.2 | 71 |
| 21.7 | 8 |
| 22.0 | 13 |
| 22.2 | 7 |
| 22.4 | 7 |
| 22.8 | 3 |
| 23.1 | 5 |
| 23.5 | 9 |
| 23.9 | 10 |
| 24.5 | 7 |
| 24.8 | 3 |
| 25.1 | 9 |
| 25.4 | 5 |
| 25.9 | 5 |
| 26.2 | 8 |
| 26.4 | 4 |
| 26.6 | 4 |
| 27.2 | 9 |
| 27.7 | 3 |
| 28.0 | 2 |
| 28.4 | 2 |
| 28.7 | 2 |
| 29.6 | 2 |
| 30.3 | 4 |

Example 2. Method of Manufacture for AV-105 Form B
Following the prophetic example, this is the Recrystallization Protocol for Form B: AV-105 (basis material, 1.00 equiv.) is dissolved in MeOH (11.5 vol.) at 40-45° C. and passed through a 0.45-μm in-line filter. The system is rinsed with MeOH (0.5 vol.) and the temperature adjusted to 30-40° C. The mixture is seeded with AV-105 Form B (1.0 wt % slurry in 0.020 vol. MeOH) with agitation at 30-40° C. for 1 hour. The temperature is adjusted to −10±3° C. (target 5° C./10 min.) and held for a minimum of 8 hours before filtration.

### Example 2A. XRPD Data of AV-105 Form B

XRPD patterns were collected as described in Example 1A.

TABLE 2
Observed peaks for AV-105 Form
B for XRPD with CuKα radiation

| °2θ | Intensity (%) |
|---|---|
| 8.7 | 9 |
| 9.0 | 25 |
| 9.2 | 25 |
| 10.3 | 20 |
| 11.6 | 5 |
| 12.3 | 18 |
| 12.7 | 40 |
| 13.3 | 81 |
| 13.5 | 43 |
| 14.0 | 13 |
| 14.6 | 20 |
| 15.1 | 9 |
| 15.8 | 2 |
| 16.5 | 6 |
| 16.7 | 3 |
| 17.1 | 7 |
| 17.3 | 8 |
| 17.5 | 19 |
| 17.8 | 59 |
| 18.1 | 10 |
| 18.6 | 13 |
| 18.9 | 36 |
| 19.4 | 96 |
| 19.6 | 7 |
| 19.9 | 11 |
| 20.1 | 21 |
| 20.7 | 100 |
| 21.1 | 10 |
| 21.9 | 16 |
| 22.2 | 4 |
| 22.7 | 33 |
| 23.1 | 8 |
| 23.4 | 6 |
| 23.7 | 61 |
| 23.9 | 7 |
| 24.2 | 9 |
| 24.6 | 9 |
| 24.9 | 14 |
| 25.2 | 26 |
| 26.0 | 13 |
| 26.1 | 22 |
| 26.7 | 4 |
| 26.9 | 5 |
| 27.4 | 14 |
| 27.6 | 32 |
| 28.1 | 6 |
| 28.4 | 3 |
| 28.9 | 5 |
| 29.1 | 7 |
| 29.3 | 4 |
| 29.5 | 4 |
| 29.9 | 4 |
| 30.4 | 3 |
| 30.6 | 5 |

### Example 3. Form B is the Thermodynamically More Stable Form

As discussed above, it is desirable to obtain thermodynamically stable compounds to assist local PET imaging centers in carrying out an efficient, consistent conversion of AV-105 to ¹⁸F-Florbetapir suitable for human administration.

Experiments were performed to determine which of Forms A and B are most thermodynamically stable. Long term slurry and DSC experiments were performed. Based on the experiments as described below, Form B was identified as the most thermodynamically stable form.

### Long Term Slurry

Samples of starting material were suspended in specified solvents and triturated at specified temperatures. After approximately 24 hours, suspensions were transferred into Spin-X centrifuge tubes equipped with solids 0.45-μm nylon filters and centrifuged. Solids separated were resuspended in fresh solvents and the agitation was continued for a total of 2 weeks. Solids were isolated as described above and analyzed by XRPD.

Measured aliquots of supernatants isolated from the solids were placed in pre-weighed TGA pans for evaporation. Once solvents were observed to have evaporated to dryness, the pans were re-weighed, and the equilibrium solubilities were calculated based on weights of the remaining solids and volumes of the corresponding aliquots.

Unless otherwise specified, solids of AV-105 were composed of Form A and were agitated in specified solvents at specified temperatures. Solvents were replaced after approximately 24 hours, where possible. After ˜2 weeks, solids were separated from supernatants via centrifugation with filtration and analyzed by XRPD. Solubilities were evaluated as a single, small scale measurement via gravimetric method using supernatants separated from the solids. Organic solvents used are anhydrous. Water activities provided in table do not account for contribution of water in starting materials and ambient RH. Approximate solvent ratios are expressed in % by volume. Temperatures and duration of experiments are approximate. The results are depicted in Table 3 below.

TABLE 3
Results of long term slurry experiments

| | | | XRPD Results/ | Solubility |
|---|---|---|---|---|
| Solvent System | Temperature | | observations | (mg/mL) |
| ACN/water | RT | | Form B | 2 |
| 50/50, A_{w} 0.90 | | | | |
| CPME | 2-8° | C. | Form B | 25 |
| | RT | | Form B | 47 |
| EtOAc/heptane | 2-8° | C. | Form B | 6 |
| 40/60 | RT | | Form B | 27 |
| | 35° | C. | Form B | 34 |
| EtOH | 2-8° C., with solvent | | Form B | 3 |
| | replacement | | | |
| | RT, with solvent | | Form B | 7 |
| | replacement | | | |
| | RT, with solvent | | Form B | 7 |
| | replacement (a) | | | |
| | 45° | C. | Yellow solution, | — |
| | | | no solids | |
| | 45° | C. (a) | Yellow gel | — |
| IPA | RT, with solvent | | Form B | 4 |
| | replacement | | | |
| | 45° | C. | Form B | 44 |
| IPrOAc | 35° | C. | Form B | 232 |
| | RT | | Form B | 93 |
| MeOH | Freezer | | Form B + small | 5 |
| | | | additional peaks | |
| | 2-8° C., with solvent | | Form B | 6 |
| | replacement | | | |
| | RT, with solvent | | Form B | 13 |
| | replacement | | | |
| | 45° | C. | Orange gel | — |
| MTBE | RT | | Form B | 34 |
| MIBK | 2-8° | C. | Form B | 128 |
| | RT | | Form B | 137 |
| | 35° | C. | Form B | 277 |
| MIBK/heptane | RT | | Form B | 15 |
| 40/60 | | | | |
| THF/cyclohexane | 2-8° | C. | Form B | 55 |
| 30/70 | RT | | Form B | 370 |
| Water | RT | | Form A | 0 |
| | 70° | C. | Orange gel after | — |
| | | | 1 day | |

^{a} Starting material composed a mixture of Form A/Form B

Slurrying Form A in a variety of solvent systems led to a conversion to Form B in all solvent systems tested between 2-8° C. and 45° C. The solvent conditions included anhydrous organic solvents as well as a high-water activity ACN/water mixture. Based on XRPD data all solids isolated from these experiments were consistent with Form B. These results confirm that Form B is thermodynamically the more stable form at these temperatures.

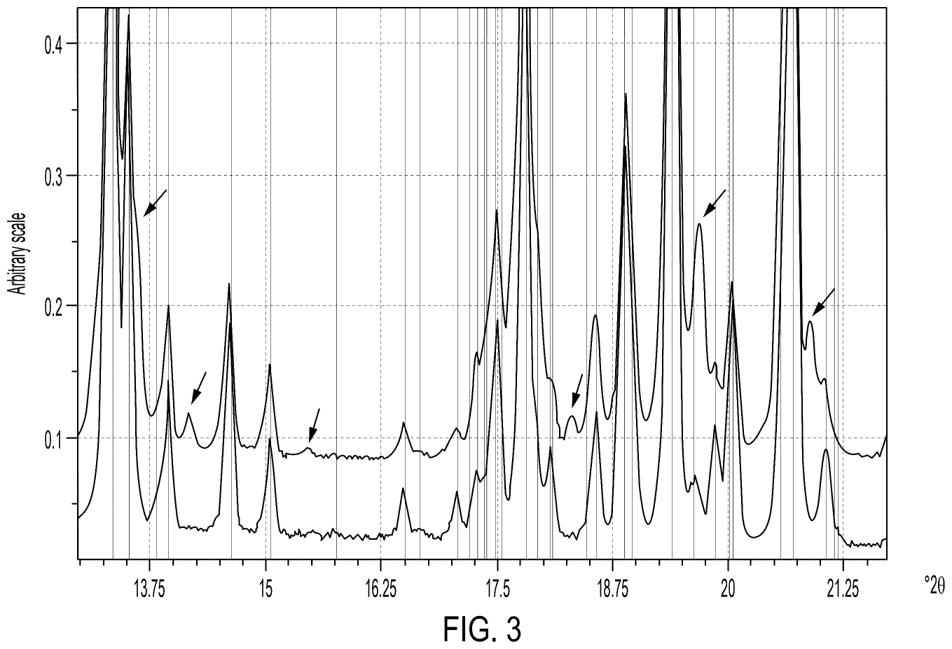

A single experiment conducted in MeOH at freezer temperatures led to Form B with small additional peaks not accounted for by the indexing solutions of Form B and Form A (e.g., see the arrows in FIG. 3) Re-slurrying the sample in MeOH at 2-8° C. for ˜5 days resulted in pure phase Form B with the additional peaks no longer observed in the XRPD pattern (e.g., see the bottom pattern in FIG. 3). This suggests that a solvate or a low temperature form may exist but is only stable at temperatures below 2-8° C.

### Differential Scanning Calorimetry (DSC)

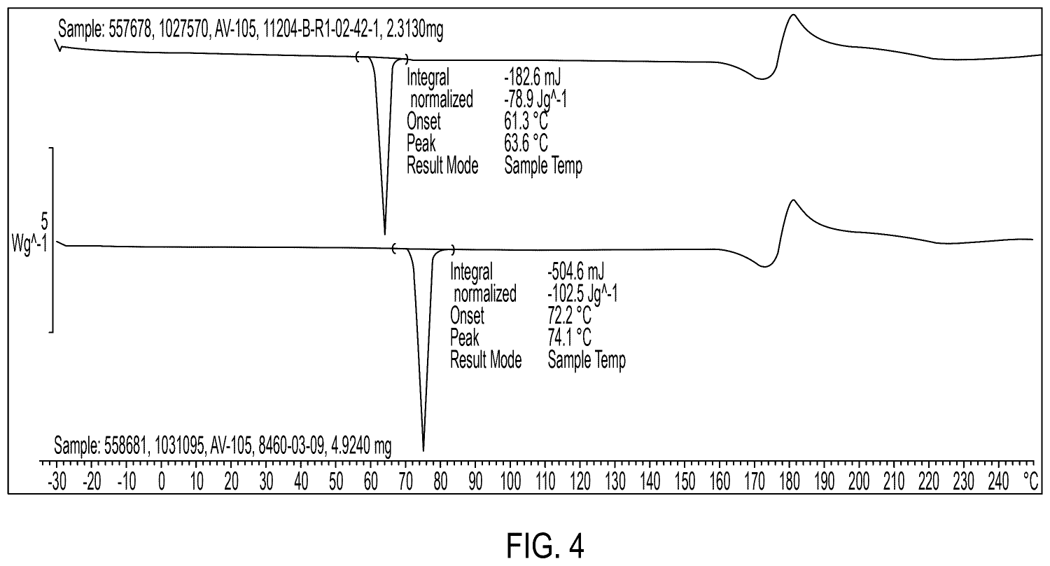

By DSC, Form B shows melting with an onset at 72.2° C. and a heat of fusion of 102.5 J/g, while the previously known Form A exhibits melts at 61.3° C. (onset) with a heat of fusion of 78.9 J/g (FIG. 4). Based on the heat-of-fusion rule (Bernstein, J. (2002). Polymorphism in Molecular Crystals. Clarendon Press, Oxford), the phase with higher melt and heat of fusion is thermodynamically more stable than the phase with lower melt/heat of fusion at all temperatures. The DSC data indicates Form B is more stable than Form A and the two forms are monotropically related. This is consistent with the screen findings, where conversion of Form A into Form B has been observed in a wide temperature range, between 2-8° C. and 45° C.

What is claimed is:

1. A compound of the formula 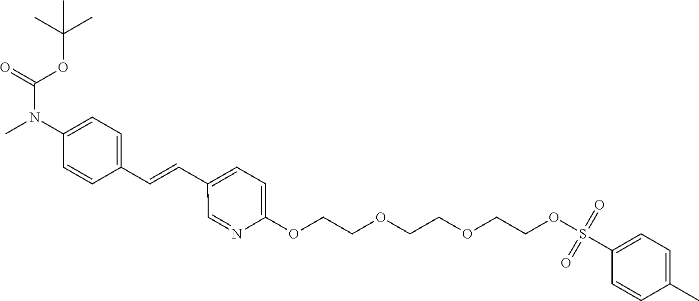 [SMILES: Cc1ccc(S(=O)(=O)OCCOCCOCCOc2ccc(/C=C/c3ccc(N(C)C(=O)OC(C)(C)C)cc3)cn2)cc1]
which is a crystalline form of AV-105 which is characterized by an X-ray powder diffraction pattern using CuKα radiation comprising a peak at diffraction angle 2-theta of 20.7° and one or more peaks selected from the group consisting of 13.3° and 19.4°; with a tolerance for the diffraction angles of ±0.2 degrees.

2. The compound of claim 1, characterized by an X-ray powder diffraction pattern using CuKα radiation comprising a peak at diffraction angle 2-theta of 20.7° and a peak at 13.3°; with a tolerance for the diffraction angles of ±0.2 degrees.

3. The compound of claim 1, characterized by an X-ray powder diffraction pattern using CuKα radiation comprising a peak at diffraction angle 2-theta of 20.7° and a peak at 19.4°; with a tolerance for the diffraction angles of ±0.2 degrees.

4. A compound of the formula 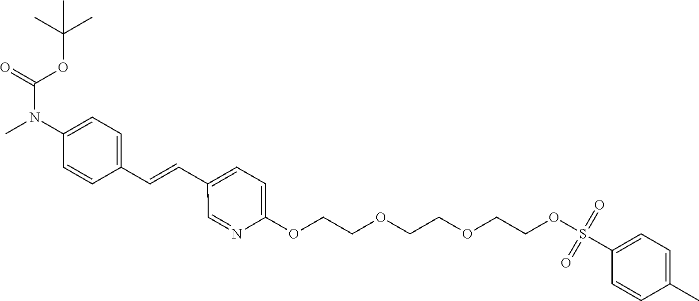 [SMILES: Cc1ccc(S(=O)(=O)OCCOCCOCCOc2ccc(/C=C/c3ccc(N(C)C(=O)OC(C)(C)C)cc3)cn2)cc1]
which is a crystalline form of AV-105 which is characterized by an X-ray powder diffraction pattern using CuKα radiation comprising a peak at diffraction angle 2-theta of 20.7° and one or more peaks selected from the group consisting of 12.7°, 13.3°, 17.8°, 19.4° and 23.7°; with a tolerance for the diffraction angles of ±0.2 degrees.

5. A compound of the formula 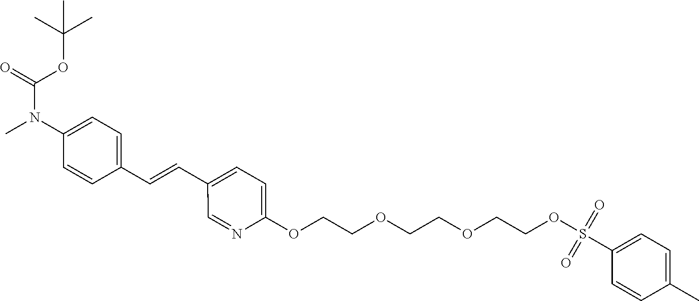 [SMILES: Cc1ccc(S(=O)(=O)OCCOCCOCCOc2ccc(/C=C/c3ccc(N(C)C(=O)OC(C)(C)C)cc3)cn2)cc1]
which is a crystalline form of AV-105 which is characterized by an X-ray powder diffraction pattern using CuKα radiation comprising two peak at diffraction angle 2-theta, wherein the two peaks are selected from the group consisting of 9.0, 9.2, 10.3, 12.7, 13.3, 13.5, 17.8, 18.9, 19.4, 20.7, 22.7, 23.7, and 27.6; with a tolerance for the diffraction angles of ±0.2 degrees.

6. A compound of the formula 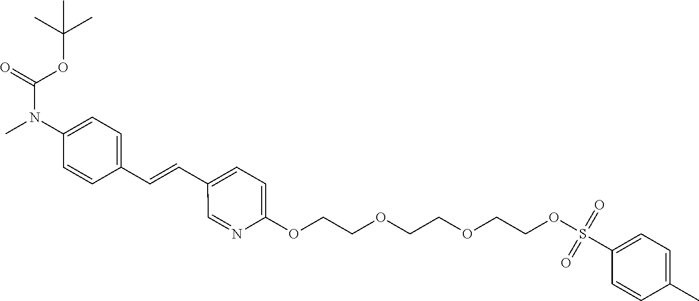 [SMILES: Cc1ccc(S(=O)(=O)OCCOCCOCCOc2ccc(/C=C/c3ccc(N(C)C(=O)OC(C)(C)C)cc3)cn2)cc1]
which is a crystalline form of AV-105 and is characterized by an X-ray powder diffraction pattern using CuKα radiation comprising a peak at a diffraction angle 2-theta of 13.3.° and at least one additional peak selected from the group consisting of 13.5°, 9.2° and 19.4°; with a tolerance for the diffraction angles of ±0.2 degrees.

7. The compound of claim 6, characterized by an X-ray powder diffraction pattern using CuKα radiation comprising a peak at diffraction angle 2-theta of 13.3° and a peak at 13.5°; with a tolerance for the diffraction angles of ±0.2 degrees.

8. The compound of claim 6, characterized by an X-ray powder diffraction pattern using CuKα radiation comprising a peak at diffraction angle 2-theta of 13.3° and a peak at 19.4°; with a tolerance for the diffraction angles of ±0.2 degrees.

9. A compound of the formula 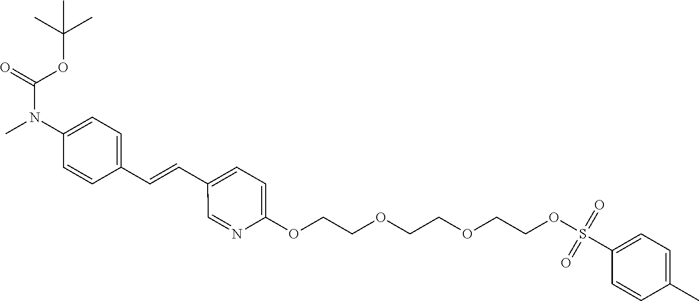 [SMILES: Cc1ccc(S(=O)(=O)OCCOCCOCCOc2ccc(/C=C/c3ccc(N(C)C(=O)OC(C)(C)C)cc3)cn2)cc1]
which is a crystalline form of AV-105 and is characterized by an X-ray powder diffraction pattern using CuKα radiation comprising peaks as depicted in FIG. 2.

10. The compound of claim 1 for use as a precursor for ¹⁸F-florbetapir.

11. The compound of claim 1 for use as a precursor for ¹⁸F-florbetapir therapy.

12. The compound of claim 1 for use as a precursor for use in PET diagnostic imaging.

13. A method of making ¹⁸F-florbetapir comprising reacting the compound of claim 1 with an ¹⁸F source.

14. A method of preparing the compound of claim 1 via recrystallization.

15. The use of the compound of claim 1 for the manufacture of a precursor to a medicament for the treatment or diagnosis of a disease or condition selected from Alzheimer's Disease and a disease associated with build-up of amyloid-beta.

16. A pharmaceutical composition comprising the compound of claim 1 and one or more pharmaceutically acceptable excipients.
